(19) United States
(12) Patent Application Publication
(10) Pub. No.: US 2004/0071776 A1
(43) Pub. Date: Apr. 15, 2004

(54) Porous plymeric biomaterials, preparation method and uses

(76) Inventors: Vincent Boudy, Paris (FR); Alexandre Laurent, Courbevoie (FR); Denis Labarre, Villebon (FR); Jean-Claude Chaumeil, St Michel sur Orge (FR)

Correspondence Address: Welsh & Katz, 22nd Floor, 120 South Riverside Plaza, Chicago, IL, 60606, US

(21) Appl. No.: 10/432,389

(22) PCT Filed: Nov. 19, 2001
(86) PCT No.: PCT/FR01/03623

(30) Foreign Application Priority Data
Nov. 22, 2000 (FR) 00/15065

(51) Int. Cl. A61K 9/14

(52) U.S. Cl. 424/486; 424/488

(57) ABSTRACT

The invention concerns porous polymeric biomaterials containing a porous polymeric matrix optionally filled with biological and/or chemical active agents, the method for preparing same and their uses, in particular as implant.

The present invention relates to porous polymeric biomaterials containing a porous polymeric matrix optionally filled with biological and/or chemical active agents, their method of preparation and their uses, in particular as an implant.

The use of resorbable or nonresorbable biomaterials is frequent in the medical field. These biomaterials can be provided in various forms and can be used, for example, for producing therapeutic vascular occlusions (embolizations), for cellular reconstruction, the treatment of gastroesophageal reflux, urinary incontinence or for the reduction of wrinkles.

Accordingly, for the production of therapeutic vascular occlusions, there has already been proposed, in particular in Patent Application FR-A-2 676 927, the use of microbeads composed of a hydrophilic acrylic copolymer coated with a cell adhesion promoting agent such as, for example, collagen, gelatin, glucosaminoglycans, fibronectin, lectins, etc. The acrylic copolymer constituting these microbeads preferably contains one or more monomers carrying a cationic charge, so as to initiate and improve cell adhesion onto the microbeads at the embolization site. Moreover, during the synthesis of these microbeads, and in order to increase the stability thereof, a crosslinking agent may be added in order to crosslink the adhesion agent coating the microbeads.

Again for the production of embolizations, there has also been proposed, in particular in Patent Application FR-A-2 784 580, microspheres comprising crosslinked polyvinyl alcohol, it being possible for said microspheres also to comprise a cell adhesion promoting agent and to be optionally impregnated with an active ingredient such as an antiangiogenic or anti-inflammatory agent. However, with this type of microspheres, it is not possible to control the release of the incorporated active principle, that is to say the delayed or prolonged release thereof.

On the other hand, International Application WO 99/44643 describes a method of treating gastroesophageal reflux in which biocompatible cationic hydrophilic microparticles are used which comprise a cell adhesion promoting agent and optionally an active principle carrying an anionic charge capable of covalently binding to said cationic microparticles. However, the use of this type of microparticles is limited to the incorporation of active principles having an anionic charge and does not make it possible to control their release either.

Finally, it has also already been proposed to coat cationic embolization microspheres on which an anionic active principle (indomethacin) had been grafted, these microspheres being coated by means of a coating polymer such as ethyl cellulose (Boudy et al., J. Pharm. Clin., 1999, 18, 21-23).

However, this technique causes modification of the physicochemical properties of the microspheres (shape, mechanical properties, surface for exchange between the biomaterial and the biological medium, etc) and does not make it possible to arrive at a controlled release of the active principle, the coating not having a significant action on the kinetics of release thereof.

However, the problem of controlling the release of biological and/or chemical active agents from a biomaterial is fundamental since these active agents should be able to be retained by the biomaterial for a period appropriate for the implantation of said material at the site where the subsequent release of the active principle is envisaged.

For example, in the context of the production of embolizations by means of microspheres, the active principle, which may be in particular an anti-inflammatory agent, should be able to remain in the microspheres during the entire preparation of the solution for injection containing the microspheres and which will serve for producing the embolization, and then during the transport of these microspheres by the bloodstream up to the embolization site where it will be finally released (this period being about 2 to 10 minutes).

It is in order to overcome these problems that the inventors have developed what is the subject of the invention.

The inventors have therefore set themselves the objective of providing a biomaterial allowing the controlled release (in terms of time and quantity) of one or more biological and/or chemical active agents.

The subject of the present invention is therefore a porous biomaterial, characterized in that it consists of a hydrophilic or amphiphilic porous polymeric network (support network) whose pores contain a gelled porous polymeric network (filling network), and in which the diameter of the pores of the support network is greater than the diameter of the pores of the filling network.

The inventors have indeed demonstrated that the presence of a filling network as defined above (in which the biological and/or chemical active agent will be contained) within a support network makes it possible to control the release (delayed or prolonged release) of said active agent, without as a result modifying the physicochemical characteristics of the support network (shape, mechanical properties, surface for exchange between the biomaterial and the biological medium, etc).

According to an advantageous embodiment of the invention, the hardness of the support network is greater than the hardness of the filling network, because the support network has a solidity provided by covalent bonds, whereas the filling network has a solidity provided by ionic interaction.

According to the invention, the support network may consist of one or more resorbable or nonresorbable polymers.

Among the polymers which can be used as support network, there may be mentioned in particular polyepsilon caprolactones, polymers and copolymers of lactic and glycolic acid, albumin, casein, crosslinked gelatins, polyanhydrides, cellulose esters and ethers, acrylic and methacrylic polymers such as acrylates and methacrylates such as, for example, polyhydroxyethyl methacrylate and its derivatives, substituted or unsubstituted polyacrylamides such as poly-(N-acryloyl2-amino-2-hydroxymethyl-1,3-propanediol) and its derivatives (TRISACRYL®), poly-(n-2-hydroxypropyl methacrylamide) and its derivatives, polyvinyl alcohols and polyurethanes.

Among the acrylic polymers, there may be mentioned most particularly the polymers derived from acrylic copolymers modified or otherwise with ionized or ionizable functional groups such as (C₁-C₄)alkylamino and (C₁-C₄)alkylamino(C₁-C₄)alkyl groups such as for example the diethylaminoethyl (DEAE) group.

Preferably, the support network of the biomaterial in accordance with the invention is a porous microsphere consisting of acrylic copolymers modified with DEAE groups. Such microspheres are for example sold under the trade name DEAE-TRISACRYL® by the company BIOSEPRA.

According to the invention, the filling network may consist of one or more resorbable or nonresorbable polymers.

Among the polymers which can be used as filling network, there may be mentioned in particular alginates, pectins, hyaluronic acid, carrageenans, agarose, agaropectins, amyloses, amylopectins, arabinogalactans, cellulose and its derivatives such as for example methyl cellulose and ethyl cellulose, chitosan, gum tragacanth, gum arabic, guar gum, xanthans, dextrans, collagen and gelatins.

Shrewdly and according to a particular embodiment of the invention, the nature of the polymers which can be used as filling network may be specifically chosen according to the nature of the enzymes which may be present at the site of implantation of the biomaterial, so that they degrade the filling network in order to release its active agent.

By way of example, the polymers of the filling network can therefore also be chosen from polymers with azo bonds which will be degraded by azoreductases of bacterial origin, glucosidic polymers which will be degraded by digestive glucosidases, mixed acrylic-azo or acrylic-glucosidic polymers or alternatively polymers containing ester bonds which will be degraded by digestive esterases.

According to a preferred embodiment of the invention, the filling network is in the form of an alginate gel.

Alginates are composed of linear successions of homopolysaccharides composed of α-1,4-D-guluronan units and β-1,4-D-mannuronan units and of linear successions of heteropolysaccharides composed of units attached at the 1,4-positions of α-L-guluronic and β-D-mannuronic acids (Ullmann's Encyclopedia of Industrial Chemistry, 1998, 25, 34-40).

The properties of alginates are essentially determined by their molecular mass and by the respective proportions of the different constituent saccharide units, these proportions varying according to the species of brown algae from which they are extracted. The hardest gels are obtained from alginates containing a high proportion of α-L-guluronic acid units.

According to the invention, it is preferable to use alginates comprising from 30% to 75% of α-L-guluronic acid units.

Among these alginate gels which can be used as filling network, there may be mentioned in particular sodium alginate gels of high viscosity such as those sold under the names MANUGEL® DJX, MANUGEL® DMB and KELTONE® HVCR by the company MONSANTO.

As already indicated above, the diameter of the pores of the support network is greater than the diameter of the pores of the filling network.

The diameter of the pores of the filling network is preferably such that it allows the diffusion of molecules whose molecular mass varies between 10 daltons (Da) and 10⁶ Da approximately and still more particularly between 10² and 10⁴ Da.

According to a preferred embodiment of the invention, the filling network of the biomaterial contains at least one biological and/or chemical active agent.

The nature of the biological and/or chemical active agent(s) which may be contained in the pores of the filling network will vary according to the applications envisaged and the size of the pores of the filling network.

By way of example, there may be mentioned in particular anti-inflammatory agents, angiogenic agents, antimitotics, angiogenesis inhibitors, growth factors, vitamins, hormones, proteins, vaccines, peptides, antiseptics, antimicrobials such as antibiotics, and generally any agent for therapeutic, preventive or diagnostic use.

The biomaterial in accordance with the invention may be provided in various forms according to the applications envisaged. It may in particular be provided in the form of a film, a block, a sheet, a stick, a thread, a particle such as for example a microsphere, or in any other form suitable for its use in the biomedical field, in particular as an implant.

According to a particularly advantageous embodiment of the invention, the biomaterial in accordance with the invention is a porous microsphere consisting of acrylic copolymers modified or otherwise with ionized or ionizable functional groups chosen from (C₁-C₄)alkylamino and (C₁-C₄)alkylamino(C₁-C₄)alkyl groups, the pores of said microsphere being filled with a porous alginate gel whose pores contain at least one biological and/or chemical active agent.

The subject of the invention is also a method for preparing such a biomaterial as defined above, characterized in that it comprises the following steps:

a) the impregnation of at least one hydrophilic or amphiphilic porous polymer (support network) with an aqueous solution (A) of at least one filling polymer in the liquid state,

b) the impregnation of said hydrophilic or amphiphilic porous polymer with an aqueous solution (B) of at least one agent capable of causing said filling polymer to pass from the liquid state to the gelled state, and optionally

c) the impregnation of said hydrophilic or amphiphilic porous polymer with a composition (C) containing at least one biological and/or chemical active agent, it being possible for said impregnation to be carried out concomitantly with steps a) and b) by adding the composition (C) to the solution (A) and/or the solution (B), or separately after steps a) and b).

According to an advantageous embodiment of the method in accordance with the invention, the support network is, in a first step, impregnated with a solution (A) as defined above, and then, in a second step, with a solution (B) also as defined above, the composition (C) being added to the solution (A) and/or (B).

The concentration of filling polymer in the solution (A) preferably varies from 0.01 to 2% by weight relative to the total weight of the solution (A); it being possible for this concentration to be gradually increased during the length of operation a).

According to this method, the filling polymer is preferably chosen from collagen, gelatins and polysaccharides such as alginates, pectins, dextrans, and carrageenans.

The nature of the agent capable of causing the filling polymer to pass from a liquid state to a gelled state (gelling agent) of course depends on the nature of the filling polymer.

By way of example, and when the filling polymer is an alginate, the gelling agent is preferably chosen from multivalent ions such as calcium ions.

The quantity of gelling agent will also vary according to the quantity of filling polymer in the liquid state which it is desired to gel and also according to the hardness of the gel which it is desired to obtain.

This quantity of gelling agent is preferably between 10% and 80% by weight relative to the weight of the filling polymer to be gelled.

The impregnation steps may be optionally carried out with stirring, at a stirring rate preferably between 150 and 2 000 revolutions per minute.

It is also possible to carry out sonication of the solutions during the impregnation steps.

Moreover, it is possible to add one or more surfactants to the aqueous solutions (A) and/or (B), and to the composition (C), in order to increase the wettability of the hydrophilic or amphiphilic porous polymer and thus facilitate the impregnation thereof with the filling polymer. These surfactants may be chosen from anionic, cationic, nonionic and amphoteric surfactants, nonionic surfactants being particularly preferred.

The temperature at which the impregnation operations are carried out varies in general from 20° C. to 90° C. approximately.

The duration of each of the impregnation operations is variable and is preferably between 1 minute and 24 hours approximately.

More particularly, step a) is preferably performed for a period between 1 and 24 hours; step b) is preferably performed for a period between 2 and 24 hours and step c), when it is carried out separately from steps a) and b), is preferably performed for a period between 12 and 48 hours.

Between each impregnation step, the support network may be optionally rinsed, preferably with water.

When the preparation of the biomaterial is complete, it is recovered and dried according to conventional separation and drying techniques (filtration, sieving, etc; air drying optionally on a fluidized bed, lyophilization, infrared radiation, etc).

The biomaterial filled or otherwise with biological and/or chemical active agents thus obtained may be provided in various forms corresponding to the form of the starting hydrophilic or amphiphilic porous polymer (beads, microspheres, sheets, sticks, films, etc) and may be used, in particular in the biomedical field, as an implant (biomaterial not filled with active agent) or as a device for the controlled release of at least one biological and/or chemical active agent.

By way of example, this biomaterial may be used in particular for the manufacture of vaccination devices, embolization devices, tissue reconstruction devices, bioactive implants, etc.

It may also be used for the manufacture of medical devices or of compositions, in particular of pharmaceutical, cosmetic, dermatological, dietetic or veterinary compositions.

In particular, the biomaterial in accordance with the invention may be advantageously used for the preparation of solutions for injection for intratissue or intravascular implantation.

The subject of the present invention is therefore also a solution for injection for intratissue or intravascular implantation, characterized in that it contains at least one biomaterial as defined above. When said biomaterial is provided in the form of microspheres, it may be used in particular for the preparation of a solution for injection for carrying out embolizations.

Preferably, this solution for injection contains porous microspheres consisting of acrylic copolymers modified or otherwise with ionized or ionizable functional groups chosen from (C₁-C₄)alkylamino and (C₁-C₄)alkylamino(C₁-C₄)alkyl groups such as diethylaminoethyl, the pores of said microsphere being filled with a porous alginate gel (microspheres for embolization) whose pores optionally contain at least one biological and/or chemical active agent.

In addition to the preceding features, the invention also comprises other features which will emerge from the description which follows, which refers to two examples of preparation of biomaterials in accordance with the invention, and to a comparative study of the kinetics of release of an active agent (indomethacin).

It should of course be understood, however, that these examples are given solely by way of illustration of the subject of the invention and do not constitute in any manner a limitation thereto.

### EXAMPLE 1

### Preparation of Porous Biomaterials Containing a Porous Filling Network

Acrylic microspheres sold under the name DEAE-TRISACRYL® by the company Biosepra were rinsed with distilled water and then drained by filtering the solution of microspheres on an 80 μm nylon filter using a vacuum pump.

The microspheres thus drained were then treated according to the conditions presented in table I below. In general, 1 g of microspheres, optionally dried beforehand under infrared radiation at 100° C. for 10 min, were soaked in a first solution of alginate (MANUGEL® DMB, MANUGEL® DJX or KELTONE® HVCR) having an initial alginate concentration [C1], for 1 hour, with or without stirring or sonication. The microspheres were then soaked in a second solution of the same alginate having a final alginate concentration [C2] for 24 hours without stirring.

The microspheres were then drained as above and then redispersed in water with stirring at 600 revolutions per minute, and then a solution of calcium ions was added. The whole was subjected to flash stirring for a few seconds and then was kept stirred at 200 revolutions per minute for 1 h 30 min.

Microspheres of DEAE-TRISACRYL® filled with a gelled porous filling network (Microspheres A to K of table I below) were obtained.

TABLE I

| | | | Initial | Final | | |
|---|---|---|---|---|---|---|
| | IR drying | | concentration | concentration | Concentration | |
| | before | Type of | [C1] of | [C2] of | of calcium | |
| Microspheres | impregnation | alginate | alginate (%) | alginate (%) | ions (g/l) | Remarks |
| A | No | MANUGEL ® DMB | 0.1 | 2 | 5 | No stirring |
| B | No | KELTONE ® HVCR | 0.1 | 2 | 5 | |
| C | Yes | KELTONE ® HVCR | 0.05 | 1 | 2.5 | |
| D | Yes | MANUGEL ® DJX | 0.5 | 0.5 | 3 | Sonication |
| E | Yes | MANUGEL ® DJX | 0.5 | 0.5 | 5 | Mechanical stirring |
| F | Yes | MANUGEL ® DJX | 1 | 1 | 25 | at 200 |
| G | Yes | MANUGEL ® DJX | 1 | 1 | 10 | revolutions/minute |
| H | No | MANUGEL ® DMB | 0.01 | 0.1 | 0.5 | No stirring |
| I | Yes | MANUGEL ® DJX | 0.3 | 0.75 | 3 | Sonication |
| J | Yes | MANUGEL ® DJX | 0.5 | 0.5 | 3 | |
| K | Yes | MANUGEL ® DJX | 0.01 | 1.31/3 | 50 | No stirring |

### EXAMPLE 2

### Preparation of Biomaterials Filled with Indomethacin

The microspheres A to K obtained above in example 1 were then impregnated by immersing in an indomethacin solution at 5 g/l for 12 to 48 hours in order to obtain microspheres filled with indomethacin.

### EXAMPLE 3

### Comparative Study of the Kinetics of Indomethacin Release

The microspheres H, I, J and K in accordance with the invention, filled with indomethacin and as prepared above in example 2, were used in this study, and also DEAE-TRISACRYL® M microspheres having simply undergone a step of impregnation in an indomethacin solution at 5 g/l, under the conditions described above in example 2 (control microspheres: MT).

The control microspheres differ from the microspheres in accordance with the invention in that the indomethacin is directly contained in the pores of the support network instead of being contained in the pores of the filling network (alginate gel).

The kinetics of indomethacin release were studied and compared for each of these microspheres. This test was performed according to the European Pharmacopeia IIIrd edition standards (test of dissolution in an apparatus with revolving paddles), under conditions such that the indomethacin released in solution does not prevent the release of the indomethacin still contained in the microspheres.

To do this, 1 g of microspheres was suspended in 800 ml of a physiological solution containing 0.9% NaCl, with stirring. Samples were collected at regular intervals in order to assay the released indomethacin by UV spectrophotometry.

The results obtained are presented in table II below, the quantity of indomethacin released being expressed as a percentage of the total quantity of indomethacin contained at to in the microsphere:

TABLE II

| Time | | | | | | | | | | | |
|---|---|---|---|---|---|---|---|---|---|---|---|
| (min) | 0 | 1 | 2 | 3 | 4 | 5 | 6 | 7 | 8 | 9 | t 50% |
| H(%) | 0 | 31 | 42 | 52 | 61 | 67 | 71 | 75 | 78 | 88 | 2′48 |
| I(%) | 0 | 35 | 34 | 38 | 43 | 58 | 59 | 58 | 59 | 99 | 4′30 |
| J(%) | 0 | 20 | 29 | 36 | 45 | 53 | 57 | 61 | 61 | 94 | 4′36 |
| K(%) | 0 | 14 | 28 | 43 | 51 | 58 | 64 | 68 | 71 | 100 | 3′48 |
| MT(%) | 0 | 39 | 57 | 66 | 74 | 79 | 81 | 84 | 86 | 95 | 1′36 |

These results show that the microspheres H to K in accordance with the invention make it possible to delay the release of indomethacin compared with the control microspheres. On the other hand, these results show that by varying the operating conditions for preparing these microspheres (nature and concentration of the alginate, concentration of calcium ions), it is possible to vary the kinetics of indomethacin release and thus control its release.

1. A porous biomaterial, characterized in that it consists of a hydrophilic or amphiphilic porous polymeric network (support network) whose pores contain a gelled porous polymeric network (filling network), and in which the diameter of the pores of the support network is greater than the diameter of the pores of the filling network.

2. The biomaterial as claimed in claim 1, characterized in that the hardness of the support network is greater than the hardness of the filling network.

3. The biomaterial as claimed in claim 1 or 2, characterized in that the support network consists of one or more resorbable or nonresorbable polymers.

4. The biomaterial as claimed in claim 3, characterized in that the polymers which can be used as support network are chosen from polyepsilon caprolactones, polymers and copolymers of lactic and glycolic acid, albumin, casein, crosslinked gelatins, polyanhydrides, cellulose esters and ethers, acrylic and methacrylic polymers, substituted or unsubstituted polyacrylamides, polyvinyl alcohols and polyurethanes.

5. The biomaterial as claimed in claim 4, characterized in that the acrylic polymers are chosen from those consisting of acrylic copolymers modified or otherwise with ionized or ionizable functional groups chosen from (C₁-C₄)alkylamino and (C₁-C₄)alkylamino(C₁-C₄)alkyl groups.

6. The biomaterial as claimed in claim 5, characterized in that the functional groups are diethylaminoethyl groups.

7. The biomaterial as claimed in claim 6, characterized in that said support network is provided in the form of a porous microsphere consisting of acrylic copolymers modified with diethylaminoethyl groups.

8. The biomaterial as claimed in any one of the preceding claims, characterized in that the filling network consists of one or more resorbable or nonresorbable polymers.

9. The biomaterial as claimed in claim 8, characterized in that the polymers which can be used as filling network are chosen from alginates, pectins, hyaluronic acid, carrageenans, agarose, agaropectins, amyloses, amylopectins, arabinogalactans, cellulose and its derivatives, chitosan, gum tragacanth, gum arabic, guar gum, xanthans, dextrans, collagen and gelatins.

10. The biomaterial as claimed in claim 9, characterized in that the filling network is an alginate gel comprising from 30% to 75% of α-L-guluronic acid units.

11. The biomaterial as claimed in any one of the preceding claims, characterized in that the filling network contains at least one biological and/or chemical active agent.

12. The biomaterial as claimed in claim 11, characterized in that the biological and/or chemical active agent is chosen from anti-inflammatory agents, angiogenic agents, antimitotics, angiogenesis inhibitors, growth factors, vitamins, hormones, proteins, vaccines, peptides, antiseptics, antimicrobials such as antibiotics.

13. The biomaterial as claimed in any one of the preceding claims, characterized in that it is in the form of a film, a block, a sheet, a stick, a thread or particles such as microspheres.

14. A porous biomaterial, characterized in that it consists of a porous microsphere consisting of acrylic copolymers modified or otherwise with ionized or ionizable functional groups chosen from (C₁-C₄)alkylamino and (C₁-C₄)alkylamino(C₁-C₄)alkyl groups, the pores of said microsphere being filled with a porous alginate gel whose pores contain at least one biological and/or chemical active agent.

15. A method for preparing a biomaterial as defined in any one of the preceding claims, characterized in that it comprises the following steps:
a) the impregnation of at least one hydrophilic or amphiphilic porous polymer (support network) with an aqueous solution (A) of at least one filling polymer in the liquid state,
b) the impregnation of said hydrophilic or amphiphilic porous polymer with an aqueous solution (B) of at least one agent capable of causing said filling polymer to pass from the liquid state to the gelled state, and optionally
c) the impregnation of said hydrophilic or amphiphilic porous polymer with a composition (C) containing at least one biological and/or chemical active agent, it being possible for said impregnation to be carried out concomitantly with steps a) and b) by adding the composition (C) to the solution (A) and/or the solution (B), or separately after steps a) and b).

16. The method as claimed in claim 15, characterized in that the support network is, in a first step, impregnated with the solution (A), and then, in a second step, with the solution (B), the composition (C) being added to the solution (A) and/or (B).

17. The method as claimed in claim 15 or 16, characterized in that the concentration of filling polymer in the solution (A) preferably varies from 0.01 to 2% by weight relative to the total weight of the solution (A).

18. The method as claimed in any one of claims 15 to 17, characterized in that the filling polymer in the liquid state is an alginate and in that the agent capable of causing said filling polymer to pass from a liquid state to a gelled state is chosen from multivalent ions, preferably calcium ions.

19. The method as claimed in any one of claims 15 to 18, characterized in that the aqueous solutions (A) and/or (B) and/or the composition (C) contain at least one surfactant.

20. The use of a biomaterial as defined in any one of claims 1 to 14 as implant or device for the controlled release of at least one biological and/or chemical active agent.

21. A device for the controlled release of at least one biological and/or chemical active agent, characterized in that it comprises at least one biomaterial as defined in any one of claims 1 to 14.

22. A composition, characterized in that it contains at least one biomaterial as defined in any one of claims 1 to 14.

23. A solution for injection for intratissue or intravascular implantation, characterized in that it contains at least one biomaterial as defined in any one of claims 1 to 14.
